(19) United States
(12) Patent Application Publication
(10) Pub. No.: US 2019/0021901 A1
(43) Pub. Date: Jan. 24, 2019

(54) Mandibular Adjustment Device for Sleep Apnea and Snoring Treatment

(71) Applicant: Kelly M. LeBlanc, DDS, PC, Spring, TX (US)

(72) Inventors: Kelly M. LeBlanc, Magnolia, TX (US); Richard A. Beck, Houston, TX (US)

(21) Appl. No.: 15/655,539

(22) Filed: Jul. 20, 2017

(51) Int. Cl. A61F 5/56; A61C 7/36

(52) U.S. Cl. CPC A61F 5/566; A61C 7/36

(57) ABSTRACT

A Mandibular Advancement Device (MAD) is disclosed which is useful for the treatment of snoring and Obstructive Sleep Apnea (OSA). The improved MAD design comprises upper and lower splints which can be set at an advanced protrusive position (as desired for snoring/OSA therapy), but which also permits some degree of lateral mandibular movement. Further, the amount of protrusive mandibular advancement is easily adjustable without the need of a dentist or special tools. Setting the protrusive position while permitting some lateral movement is accomplished using connector blocks connected to the splints. The connector blocks comprise linear protrusions which can be laterally slid together to interlock and hold a horizontal and vertical position of the upper and lower splints.

### FIELD OF THE INVENTION

The present invention relates to a mandibular adjustment device for the treatment of snoring and sleep apnea, and in particular to a mechanism for easily locking the upper and lower splints of the device together.

### BACKGROUND

As explained in U.S. Patent Application Publication 2014/0020691, one of the most widespread sleep disorders is snoring, an affliction with significant health and social consequences. Snoring is a sound produced by the vibration of soft tissue in a patient's upper airway caused by breathing obstruction during sleep. There are many factors associated with snoring including, but not limited to: heredity, body weight, age, gender, smoking history, tissue deformities, alcohol use, allergies, and sleep position. Commonly, the snorer, and perhaps the snorer's sleep partner, lose sleep due to the snorer's snoring. Lack of sleep can lead to daytime fatigue, a compromised immune system, poor mental and emotional health, irritability and lack of productivity, as well as other problems.

Obstructive Sleep Apnea (“OSA”) is a potentially lethal sleep and breathing disorder defined as the cessation of breathing for a certain length of time (10 seconds or longer) and with a certain frequency. When breathing is interrupted, the body reacts by waking enough to start breathing again. Episodes may occur hundreds of time each night, and may not fully awaken the individual, who remains otherwise unaware of the loud snoring, choking and gasping for air typically associated with OSA. The health risks of OSA include higher risks or occurrences of hypertension, heart attack, stroke, daytime somnolence, depression, fibromyalgia, cardiac arrhythmia, inefficient metabolism, loss of short term memory, weight gain, gastric reflux, high blood pressure, diabetes, severe anxiety, memory and concentration impairment, morning headache, intellectual deterioration, mood swings/temperamental behavior, insomnia, and impotence. Additionally, many OSA sufferers do not receive a sufficient amount of sleep due to repeated apnea events and arousals which act to prevent deep stage sleep, which can lead to chronic daytime exhaustion. An estimated 40 million Americans suffer from some degree of OSA, yet only a small fraction of these are currently undergoing any type of treatment.

Snoring and OSA are generally caused by blockage of the upper airway when various tissues relax, including the tongue, uvula, and soft palate. Snoring is caused by the partial obstruction of breathing during sleep, while OSA occurs when the tongue and soft palate collapse onto the back of the throat and completely block the upper airway, thereby stopping breathing and restricting the flow of oxygen.

Many techniques exist to reduce or eliminate snoring and OSA. Various types of surgery, including tracheostomy, surgery of the soft palate and oropharynx, and reconstructive surgery have been utilized in the treatment of snoring and OSA. Surgery however is costly, carries risks, and is not always effective.

Respiratory therapies for the treatment of OSA have been utilized as well, generally utilizing a ventilator type mask on the patient that supplies air/oxygen at a higher than atmospheric pressures, generally known as Continuous Positive Air Pressure systems or CPAP systems. Variations of this therapy having variable pressures have also been used, such as Bi-level Positive Air Pressure (BiPAP) or Multiple Positive Air Pressure (MPAP) systems. These systems however suffer from significant patient non-compliance, because patients often find it difficult to acclimatize to the mask and the pressures they provide. These systems can also be noisy, and can also affect the patient's sleeping partner, even to the point that the patient and the sleeping partner will sleep in separate rooms to each have an effective night of sleep. In other words, patients often dislike and therefore stop using respiratory therapies of these types.

Another class of snoring/OSA solutions includes appliances that patients wear to reposition theirs jaws to inhibit their airways from closing. Such devices are generally referred to as Mandibular Advancement Devices (MADs) or Mandibular advancement splints. A MAD treats snoring and OSA by advancing the mandible (lower jaw) forward slightly in a protrusive (generally horizontal) direction relative to the maxilla (upper jaw). This tightens the soft tissue of the upper airway to prevent obstruction during sleep. Such tightening also inhibits these tissues from vibrating as air passes over them, thus reducing snoring. MADs are generally less cumbersome than respiratory therapy systems (CPAP, BiPAP, etc.), which promotes high patient compliance. MADs may be prescribed by different types of physicians, but given that they are worn on the teeth, MADs are generally produced and fit under the direction of dentists, orthodontists and the like.

A MAD disclosed in the above-referenced '691 Publication and useable to treat snoring and OSA is shown in FIG. 1. The MAD 10 comprises an upper splint 12 configured to conform to and receive at least some of a patient's upper teeth (i.e., growing out of the upper jaw or maxilla), and a lower splint 14 configured to conform to and receive at least some of the patient's lower teeth (i.e., growing out of the lower jaw or mandible). The molding/conforming of the splints 12 and 14 to the patient's upper and lower teeth and the materials used for forming these structures are well known to those of ordinary skill in the art.

MAD 10 includes a mechanism on both of its sides (left side mechanism only shown in FIG. 1) that affixes the lower splint 14 to the upper splint 12. In so doing, the mechanism holds the patient's lower teeth (and mandible) at a set protrusive position (i.e., along horizontal direction 18) relative to the patient's upper teeth (and maxilla) when the patient bites into the MAD 10.

The mechanism includes a strap 32 to set this protrusive position, which again will tend to advance the patient's mandible forward. Strap 32 may be formed out of metal or hard plastic, and as such is able to firmly hold the set protrusive position. The strap 32 connects to brackets 20 and 40 fixed into splints 12 and 14 respectively. Brackets 20 and 40 may be affixed to or molded within the splints 12 and 14 during the splints' manufacture. Brackets 20 and 40 each include a threaded opening (not shown) for receiving threaded screws 30 and 44 respectively. Strap 32 includes openings 34 and 36 along its length. To set the protrusive position along horizontal direction 18, a screw 30 is passed through a chosen opening 34 and is tightened to the threaded hole (not shown) in bracket 20, and a screw 44 is passed through a chosen opening 36 and is tightened to a threaded hole (not shown) in bracket 40. Choice of the appropriate openings 34 and 36 along the length of the strap 32 changes the relative protrusive position of the brackets 20 and 40, and thus changes the protrusive position of the lower splint 12 (and the patient's mandible) relative to the upper splint 12 (and the patient's maxilla). As discussed further in the '691 Publication, shims (not shown) may also be connected to the horizontal face of bracket 20 to change the vertical position (along vertical direction 16) of the lower splint 14 relative to the upper splint 12, but this detail isn't shown.

### BRIEF DESCRIPTION OF THE DRAWINGS

FIG. 1 shows a prior art Mandibular Advancement Device (MAD) used to treat snoring and Obstructive Sleep Apnea (OSA).

FIG. 2 shows an improved MAD, in accordance with an example of the invention, and having connector blocks connected to upper and lower splints.

FIG. 3 shows further details of the upper and lower connector blocks.

FIG. 4 shows a jig useable to connect in perfectly parallel and planar fashion the upper connector blocks to the upper splint and to connect the lower connector blocks to the lower splint.

FIGS. 5A-5E show how the upper and lower connector blocks can be connected to connect the upper and lower splints of the MAD together and further shows how the design of the connector blocks allows for lateral movement of the lower splint with respect to the upper splint.

FIGS. 6A-6F show how the lower splint may be easily disconnected and reconnected to the upper splint to set an amount of protrusive mandibular advancement to the MAD.

FIGS. 7A-7G show how the upper and lower splints can optionally be locked at a desired protrusive mandibular advancement by affixing a second stop to the upper and/or lower splints.

### DETAILED DESCRIPTION

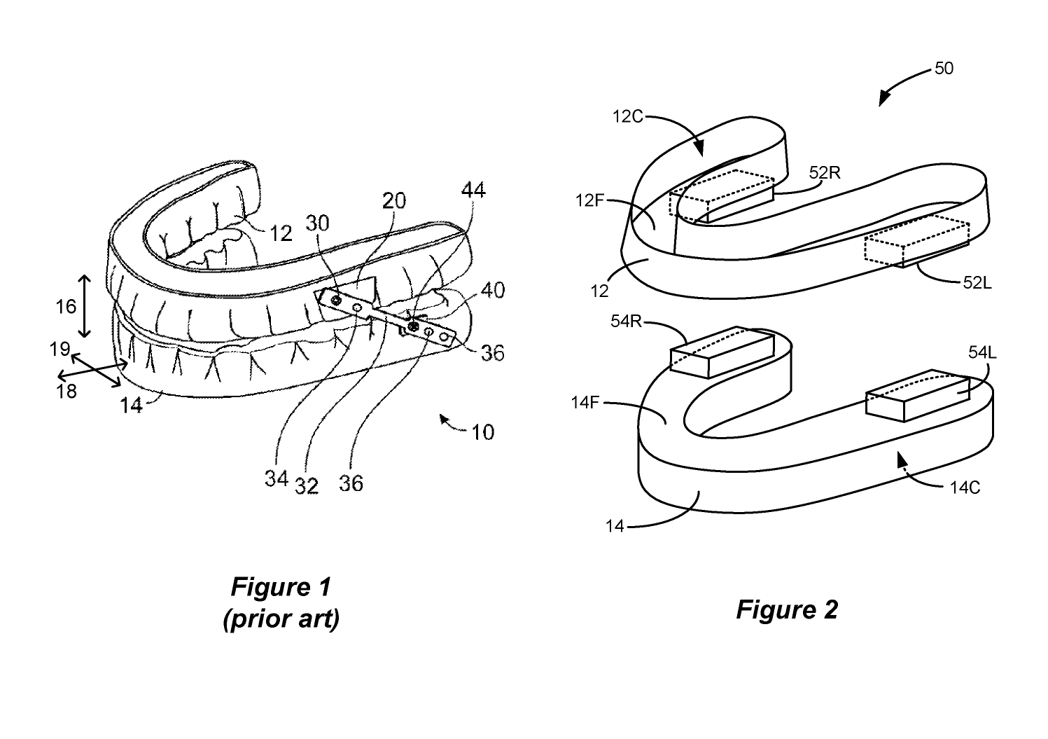

The Mandibular Advancement Device (MAD) 10 disclosed in FIG. 1 allows for protrusive positioning (advancement) of a patient's mandible, and thus, MAD 10 should be effective in treating snoring and OSA for the reasons stated earlier.

Nonetheless, the inventor sees that the design of a MAD such as 10 suffers from certain shortcomings. For one, although the amount of protrusive mandibular advancement can be adjusted in MAD 10, it is not simple to make such an adjustment, and may require special dentist tools able to remove what are likely specialty screws 30 and 44. In short, MADs such as 10 are generally considered to be adjustable only by the prescribing dentist or orthodontist. This is unfortunate, because a patient may wish to experiment with the amount of protrusive advancement. For example, a patient's sleep partner may report that the patient is still snoring despite use of MAD 10. Such a patient may wish to increase the amount of protrusive advancement to see if his sleep partner notices a reduction in his snoring. Or, a patient's snoring may be under control, but he may be sore because the MAD 10 is providing too much protrusive advancement, which can strain the ligaments of the mandible. Such a patient may wish to decrease the amount of protrusive advancement. In either case, the patient cannot easily experiment with the amount of protrusive advancement, and must instead make an appointment with his dentist or orthodontist to adjust the same.

The inventor also perceives of another shortcoming of MAD 10 in that the strap 32 holds the lower splint 14 and upper splint 12 at rigid and fixed position with respect to each other. As such, MAD 10 does not allow for lateral mandibular movement, i.e., along lateral direction 19 or left to right of the patient. This can be problematic because some patients at night are prone to grinding their teeth (bruxism), which involves lateral left/right movement of the lower teeth (mandible) relative to the upper teeth (maxilla). If a MAD doesn't permit such lateral movement, the device may feel unnatural to such “grinders,” and may be disruptive or uncomfortable causing them to discontinue using the MAD. Failing to permit lateral movement can also stress the Temporomandibular Joint (TMJ), which can cause long term soreness in many traditional MAD designs.

Accordingly, the inventor proposes a new design for a MAD 50 shown in FIG. 2, which design in particular is able to lock the upper and lower splints 12 and 14 at a set advanced protrusive position (as desired for snoring/OSA therapy), but which also permits some degree of lateral mandibular movement. Further, the amount of protrusive mandibular advancement is easily adjustable without the need of a dentist or special tools, and without the excessive bulk of other adjustable MADs designs.

As shown in FIG. 2, MAD 50 uses upper and lower splints 12 and 14, which can comprise many well-known structures, and which may generally be similar to the splints used in the prior art (FIG. 1). For example, splints 12 and 14 can be made of acrylic and formed using molds taken of a patient's upper and lower teeth. In this regard, and as one skilled in the art will appreciate, splints 12 and 14 may in reality look different from their simple appearance in FIG. 2: they may be thin and conformal to the shape of the patient's teeth; they may be of different sizes, etc. The amount of material and the size of each splint 12 and 14 may depend on the amount of dentition support required to effectively adjust the mandible position of the patient, and preferably enough material is provided to prevent placing an undue stress on the user's teeth.

Each splint 12 and 14 has a channel region 12C and 14C for accepting the patient's upper and lower teeth respectively, and flat surfaces 12F and 14F corresponding respectively to the bottoms of the upper teeth and the tops of the lower teeth. These flat surfaces 12F and 14F can be made perfectly flat (e.g., planar) when the splints 12 and 14 are formed, or may conform to the teeth and be somewhat bumpy. As shown later, flat surfaces 12F and 14F face each other when the upper and lower splints 12 and 14 are interlocked.

Connector blocks are connected to the flat surfaces 12F and 14F on the right and left sides of each of the splints 12 and 14, proximate to where a patient's right and left molar and/or pre-molar teeth would reside within the splints. Specifically, upper connector block 52R is connected to the upper splint 12 on the right (from the patient's perspective) under the patient's right upper molar and/or pre-molar teeth, upper connector block 52L is connected to the upper splint 12 on the left under the patient's left upper molar and/or pre-molar teeth, lower connector block 54R is connected to lower splint 14 on the right above the patient's right lower molar and/or pre-molar teeth, and lower connector block 54L is connected to lower splint 14 on the left above the patient's left lower molar and/or pre-molar teeth. Although the design details of the connector blocks are not shown in FIG. 2 for simplicity, the right connector blocks 52R/54R are designed to connect together as a pair, and the left connector blocks 52L/54L are designed to connect together as a pair, as will be discussed further below. Connecting these connector blocks pairs affixes the splints 12 and 14 together in a manner that sets a protrusive advancement of the patient's mandible relative to the maxilla when a patient bites into the connected splints 12 and 14.

The connector blocks can be connected to the splints 12 and 14 in different manners. As is preferable, and described in detail later, the connector blocks can be formed separately and then affixed to the splints 12 and 14. Alternatively, the connector blocks can be connected to the splints 12 and 14 by being integrally formed with the splints 12 and 14, i.e., formed at the same time that the splints are formed and out of the same material.

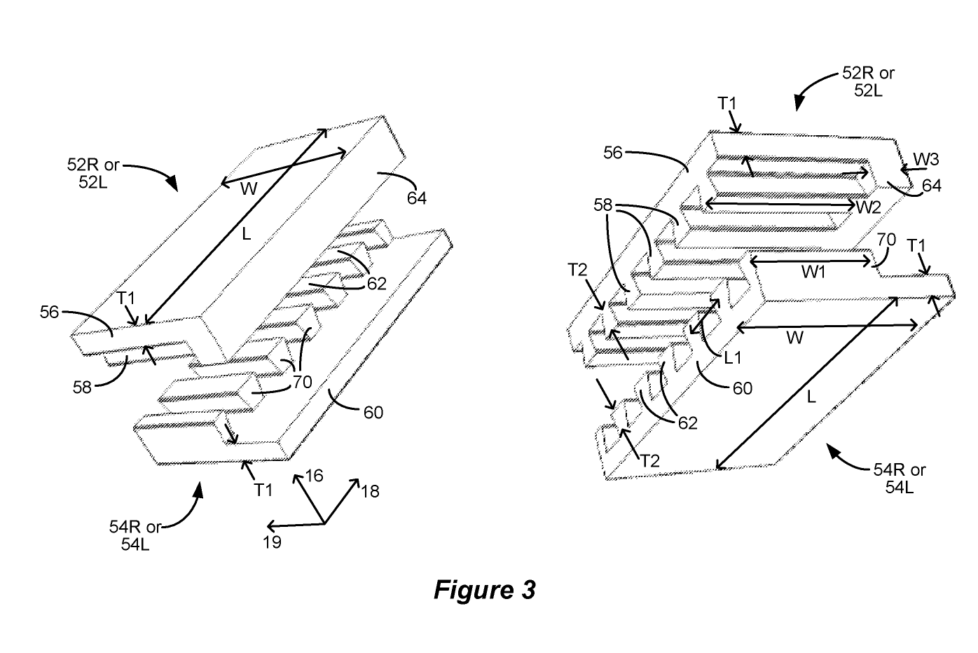

FIG. 3 shows details of the connector blocks, and specifically shows the details of a connector block pair, i.e., either right connector blocks 52R/54R or left connector blocks 52L/54L. These two pairs are preferably the same in the improved MAD 50, and hence only one pair in shown in FIG. 3 for simplicity.

The connector blocks are all generally planar in shape. Lower connector blocks 54R/54L as shown comprises a flat substrate 60 having a length L parallel to horizontal direction 18, a width W parallel to lateral direction 19, and a thickness T1 parallel to vertical direction 16. On the top of substrate 60 are linear protrusions 62, which are parallel to the lateral direction 19 and are spaced from each other in a horizontal direction 18. Thus, the linear protrusions 62 are parallel to width W, but which are shorter (W1) than width W. The linear protrusions 62 end at faces 70. Each of the protrusions 62 are spaced from each other (L1) at 3 mm for example, and may have a thickness (T2) of about 2 mm, although these values can be varied.

Upper connector blocks 52R/52L as shown comprises a flat substrate 56. It is preferable that flat substrate 56 have the same dimensions as flat substrate 60 (L, W, T1), but this isn't required in all useful designs. On the bottom of substrate 56 are linear protrusions 58, which are again are parallel to the lateral direction 19 (and width W) and are spaced from each other in a horizontal direction 18. Protrusions 58 are preferably shorter (W2) than width W, and longer than the protrusions 62 (W1), for reasons explained below. At the edge of substrate 56 is a first stop 64, which runs parallel to the length L in the horizontal direction 18 (and hence perpendicular to linear protrusions 58/62 along lateral direction 19), and has a width of W3. The thickness of the first stop 64 and protrusions 58 preferably equal the thickness T2 of the protrusions 62. Each of the protrusions 58 are spaced at the same distance (L1) as the linear protrusions 62.

Notice that the linear protrusions 62 and 58 preferably have the same trapezoidal shape in cross section (as viewed in the horizontal direction 18), with the spaces between each being shaped to mate with the protrusions on the other connector block. As will be shown below, this allows the protrusions 62 of lower connector blocks 54R/54L to be slid laterally (19) into the spaces between the protrusions 58 of upper connector blocks 52R/52L. When the connector block pairs are connected in this manner, the trapezoids in the protrusions 58 and 62 will interlock, and thus lower connector blocks 54R/54L will not be able to move in a horizontal direction 18 or a vertical direction 16 relative to the upper connector blocks 52R/52L. Cross sectional shapes for the linear protrusions 58 62 other than trapezoids can also prohibit such relative movements. For example, the protrusions could be T-shaped on the lower connector blocks 54R/54L, with interlocking with upside-down T-shapes on the upper connector blocks 52R/52L. The linear protrusions 62 and 58 may also have different cross-sectional shapes still able to interlock and prevent movement in horizontal 18 and vertical 16 directions.

While there are preferably differences in the construction and design of connector blocks 52 and 54, it does not matter in an actual implementation which is the upper and lower blocks. For example, while FIG. 3 shows connector blocks 52R/52L on the top (e.g., connectable to upper splint 14) and connector blocks 54R/54L on the bottom (e.g., connectable to lower splint 12), these could be swapped.

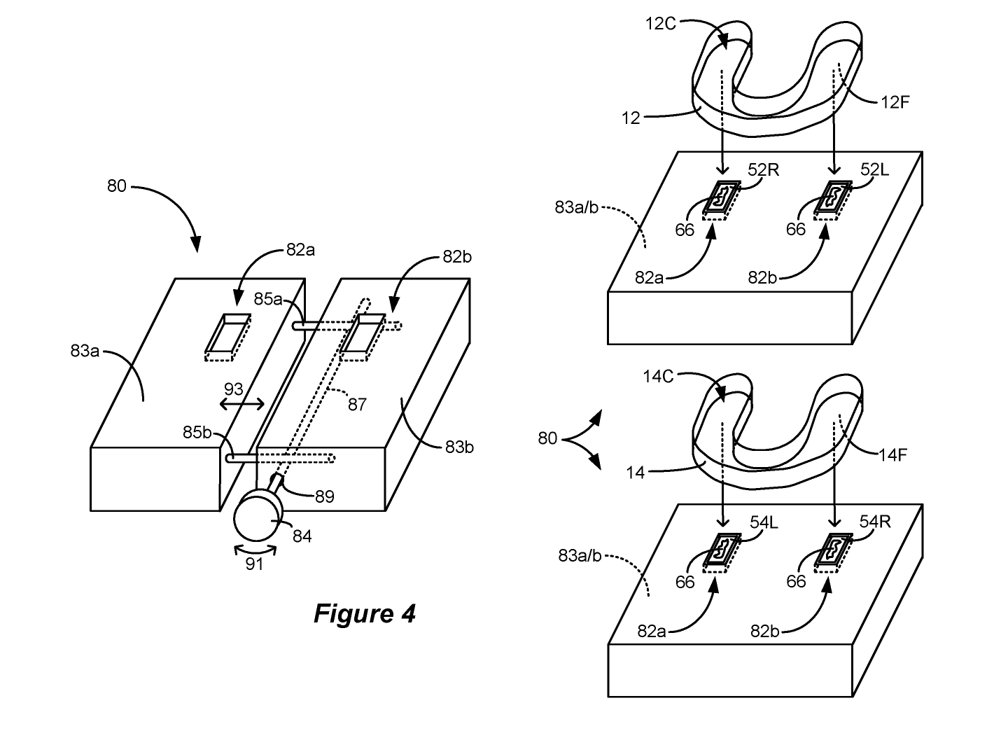

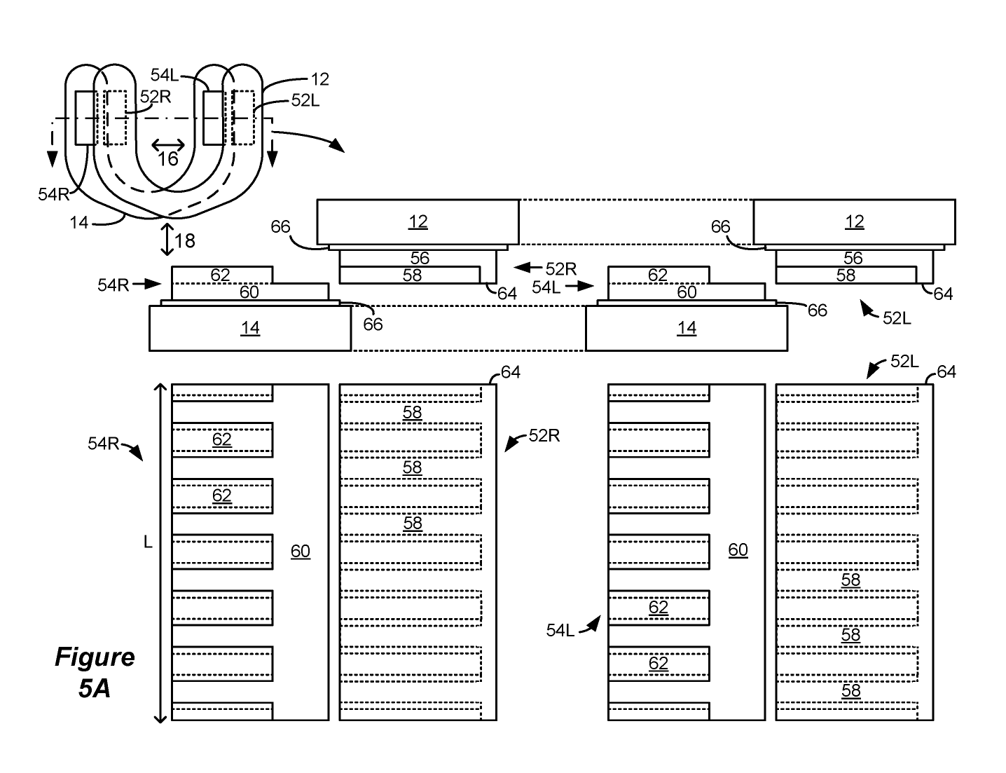

As discussed earlier, upper connector blocks 52R/52L can be connected by virtue of being integrally formed with splint 12 and connector blocks 54R/54L can be connected by virtue of being integrally formed with splint 14. However, in a preferred embodiment, the connector blocks are formed separately from the splints 12 and 14, and then are affixed thereto, as shown in FIG. 4. This is preferred because there is a forward protruded bite model taken by the dentist or orthodontist that is unique to each individual patient. The mounting of the upper and lower jaw models will determine how the connector blocks should be attached to the upper and lower splints 12 and 14.

The connectors blocks can be formed from any suitable rigid, biocompatible, FDA-approved material, which may comprise the same material used to form the splints 12 and 14 (e.g., acrylic), although other rigid plastics and metals may be used as well. The material of the connector blocks may be machinable, so that their shapes (the linear protrusions, the first stops, etc.) can be milled from otherwise solid blocks. Alternatively, the connector blocks may be produced from a mold, 3D printed, etc.

FIG. 4 shows a jig 80 that can be used to affix the connector blocks to their respective splints. The jig 80 as shown includes two different pieces 83a and 83b, which may be formed generally as solid pieces, such as stainless steel. Each piece 83a and 83b includes a recess 82a and 82b designed to hold one of the pre-formed connector blocks. Assembly of MAD 50 using jig 80 is beneficial because, as will be seen in subsequent drawings, right connector block pair 52R/54R and left connector block pair 52L/54L must be in perfectly parallel and planar alignment so that they can be laterally slid together and interlocked, a goal that recesses 82a and 82b promote. If the connector blocks are connected to the splints in any arbitrary fashion, they will not slide together and will bind.

Jig 80 is preferably adjustable to adjust the lateral spacing between the recesses 82a and 82b. Support rods 85a and 85b are affixed to piece 83a and proceed into channels (not shown) in piece 83b. An axle 87 is provided through a channel 89 in piece 83b, which channel 89 intersects with and overlays the channels in 83b that receive the support rods 85a and 85b. The axle 87 may be connected to a dial 84 that is turnable by the manufacturer of MAD 50 (e.g., the dentist, orthodontist, or an associated lab). The axle 87 and support rods 85a and 85b are preferably threaded (not shown), such that turning axle 87 (91) is translated into linear movement (93) of piece 83a relative to piece 83b. For example, axle 87 may comprise a pinion, and support rods 85a and 85b may comprise racks. In short, turning dial 84 causes the distance 93 between pieces 83a and 83b to vary. This is desirable, because different patients will have different sized jaws, and hence will have splints 12 and 14 with differing widths. Thus, jig 80 can be adjusted so that the connector blocks can be properly affixed to the flat surfaces 12F and 14F of splints 12 and 14, regardless of their size.

Connection of the connector blocks to the splints is shown to the right in FIG. 4. Notice for simplicity that the two-piece 83a/b construction of the jig 80 and its adjustability (84) is not shown; instead it is assumed that the jig 80 has already been adjusted to fit the sizes of the splints 12 and 14 in question.

At the top right, upper connector blocks 52R/52L are being connected to the upper splint 12. Connector block 52R has been placed into recess 82a, and connector block 52L has been placed into recess 82b. These connector blocks 52R/52L are placed with their flat substrates 56 facing outward toward the upper splint 12. An adhesive 66 is placed on the back of the substrates 56, which adhesive 66 material may be of the same type used to form the splints 12 and 14 (e.g., a hard-setting cold-cure acrylic). Once the adhesive 66 is placed, the flat surface 12F of upper splint 12 may then be pressed downward onto the adhesive 66 and allowed to set.

The bottom right of FIG. 4 shows connection of lower connector blocks 54R/54L to the flat surface 14F of lower splint 14. Notice here that left connector block 54L resides in recess 82a and right connector block 54R resides in recess 82b; this is necessary so that connector block pairs 52R/54R and 52L/54L will connect when the flat surfaces 12F and 14F are facing each other and connected to form the MAD 50. Furthermore, care should be given to how the connector blocks are oriented in the recesses 82a and 82b so that they will interlock as described.

Once the planar connector blocks are connected to the upper and lower splints 12 and 14, the result is that the right and left upper connector blocks 52R/52L are located in a first plane, and the right and left lower connector blocks 54R/54L are located in a second plane parallel to the first plane, once the connector block pairs 52R/54R and 52L/54L are interlocked, as discussed next.

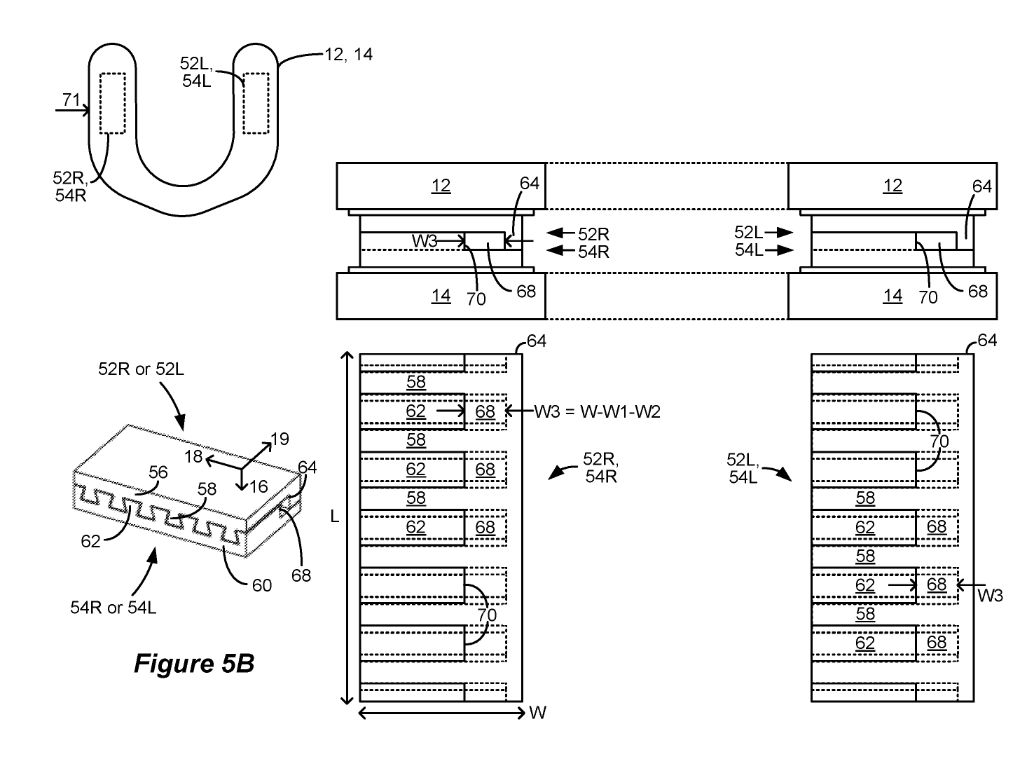

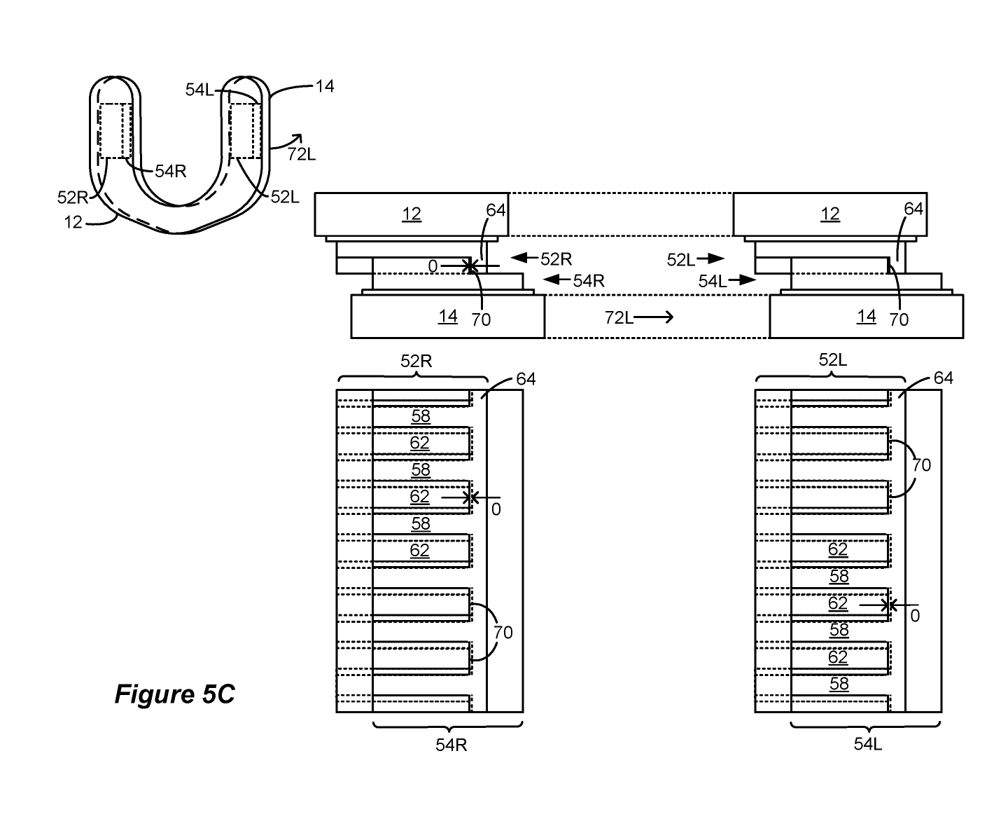

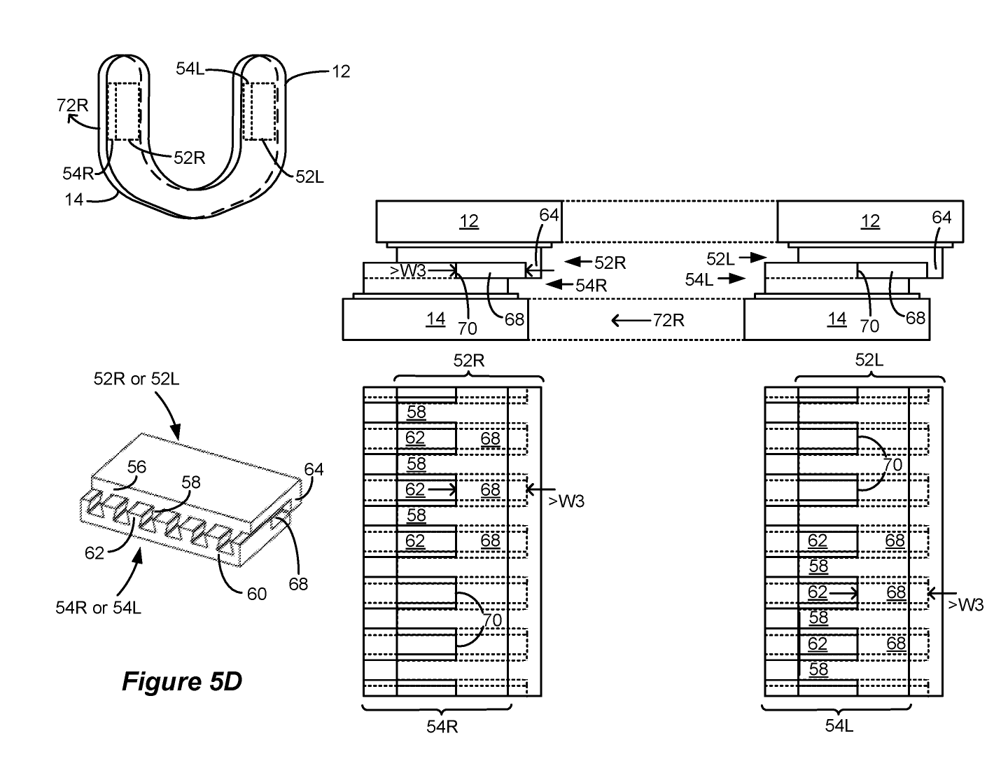

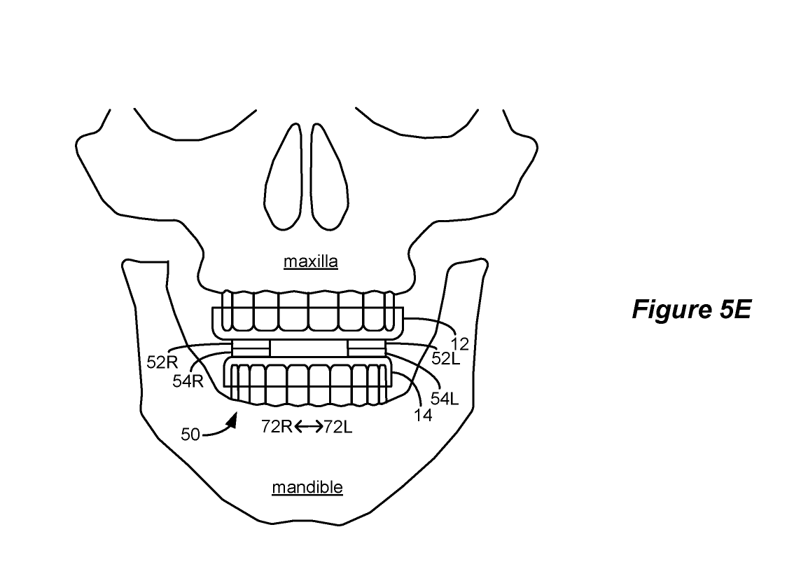

Once the connector blocks have been connected to the splints, the connector blocks can be connected together to form MAD 50, and this is shown starting with FIG. 5A. FIG. 5A shows a top down of the splints 12 and 14 (top left) as well as a top down view of the connector block pairs 52R/54R and 52L/54L at higher magnification. FIG. 5A also shows the splints and connector blocks in cross section.

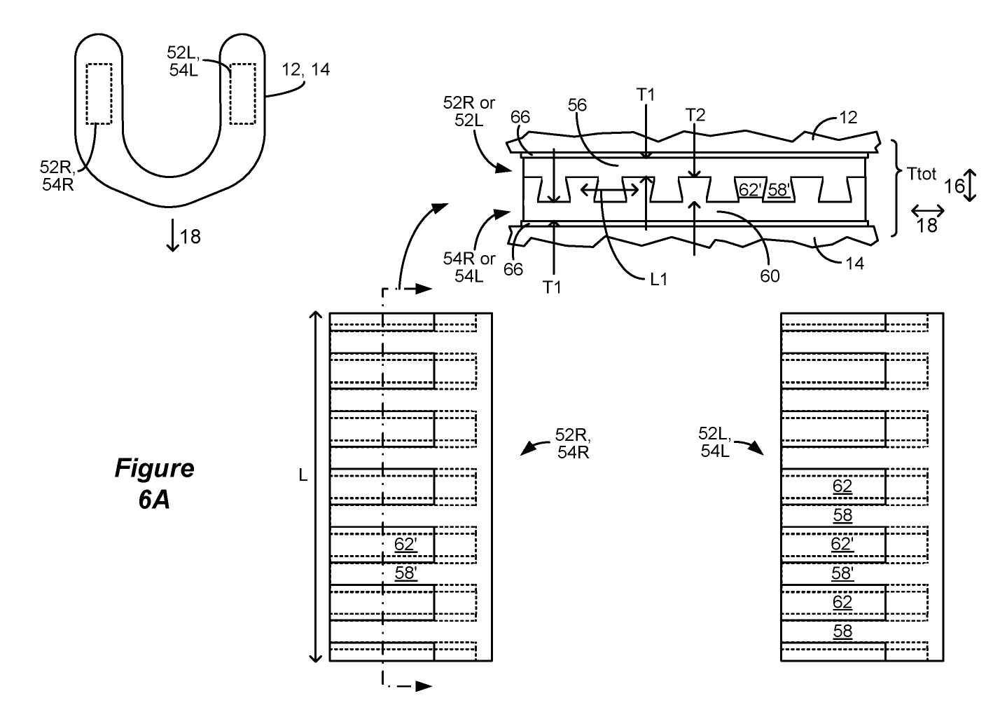

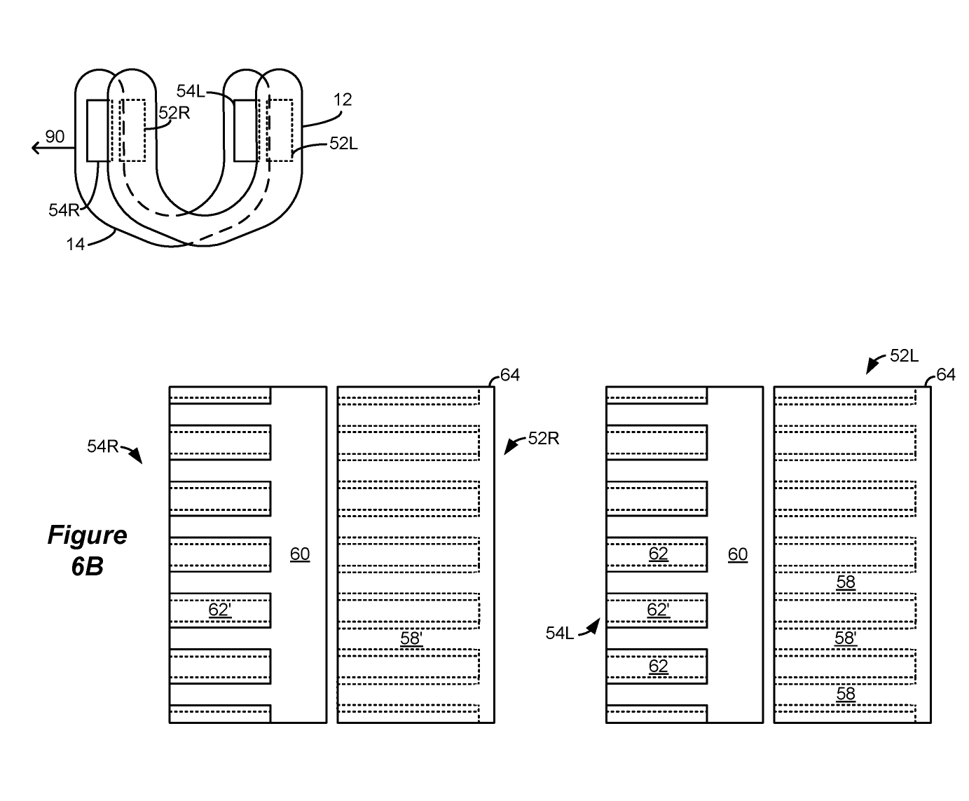

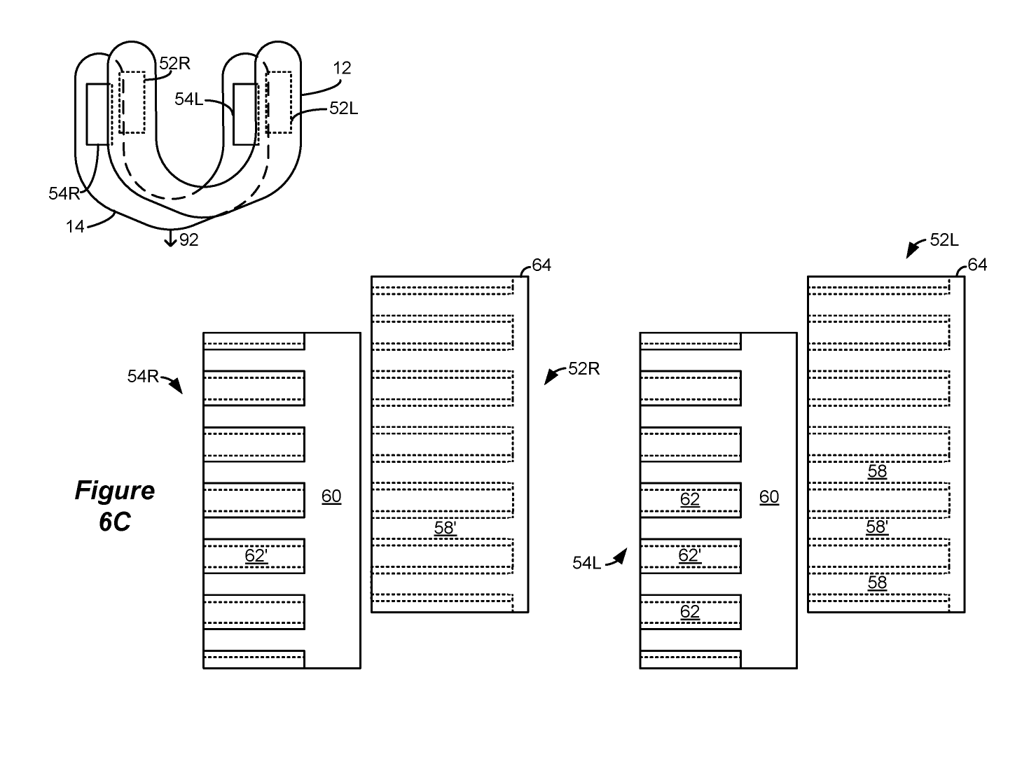

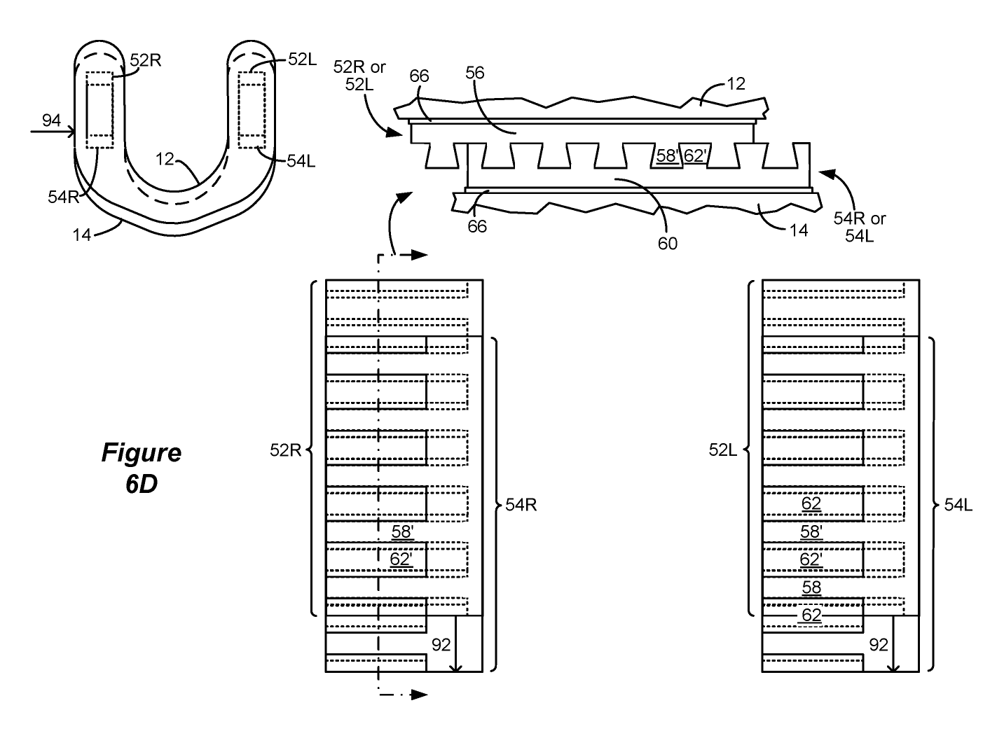

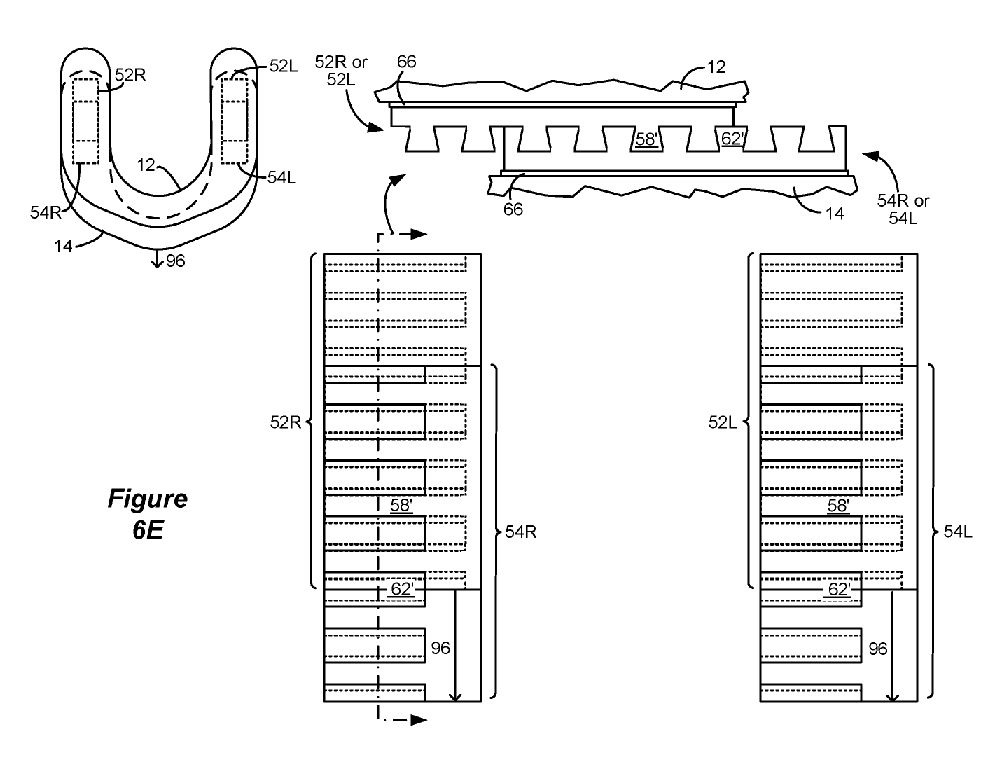

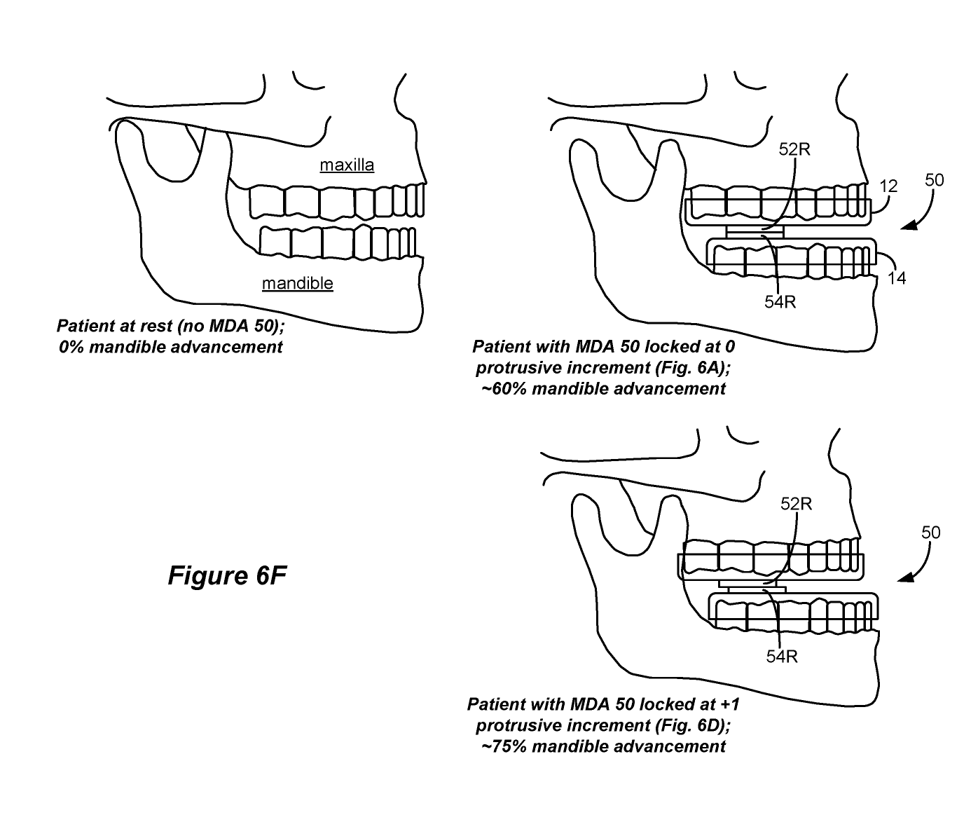

In FIG. 5A, the connector block pairs are not connected, but are in position to be connected. The lower splint 14 is shifted in a lateral direction 16 to the right (from the patient's perspective) of the upper splint 12, and the connector block pairs 52R/54R and 52L/54L are aligned such that the linear protrusions 62 of lower connector blocks 54L and 54R and the linear protrusions 58 of upper connector blocks 52L and 52R are parallel and in the same horizontal plane. Notice also in FIG. 5A that the lengths L of the connector block pairs are aligned in horizontal direction 18, although this isn't necessary, as will be described subsequently with references to FIG. 6A-6F. Positioning of the splints/connector blocks as shown in FIG. 5A occurs outside of the patient's mouth, which can be accomplished by the patient's hands.

The lower splint 14 is then moved (e.g., by the patient) in a lateral direction 16 to the right (71), as shown in FIG. 5B, with the linear protrusions 62 sliding through the spaces between linear protrusions 58, and the linear protrusions 58 sliding through the spaces between linear protrusions 62. This sliding movement continues until the widths W of the connector blocks in each pair 52R/54R and 52L/54L are aligned, such that lower connector blocks 54R/54L completely underlie upper connector blocks 52R/52L. As noted earlier, the interlocking shapes of the protrusions will keep lower connector blocks 54R/54L from moving in a horizontal direction 18 and in a vertical direction 16. At this point, the MAD 50 is constructed, and the patient may put the now-connected MAD 50 in his mouth, for example at bedtime.

However, even when the connector blocks are interlocked, the lower connector blocks 54R/54L can still slide laterally (19) with respect to the upper connector blocks 52R/52L. In this regard, notice in FIG. 5B that when the lower connector blocks 54R/54L perfectly underlie upper connector blocks 52R/52L, there is still a lateral gap 68 of width W3 between the first stops 64 of the upper connector blocks 52R/52L and the faces 70 of the linear protrusions 62 of the lower connector blocks 54R/54L. This lateral gap 68 results because the sum of the width of the linear protrusions 58 (W1; see FIG. 3) and the width of the first stops 64 (W2) is less than the width of the connector blocks (W). Width W3 of lateral gap 68 is thus W-W1-W2, and therefore can be adjusted by changing any of these values.

Lateral gap 68 permits the lower connector blocks 54R/54L to shift an additional lateral amount 72L to the left, as shown in FIG. 5C. Essentially, the linear protrusions 62 of the lower connector blocks 54R/54L can move until their faces 70 come into contact with the first stops 64. At this point, the lateral gap 68 has been closed, and its width is zero. Thus, first stops 64 prevent excessive movement of the lower connector blocks 54R/54L, and thus lower splint 14, in lateral direction 19, limiting such movement (to the left) to lateral amount 72L. Note that to prohibit left lateral movement, it is only necessary for only one of upper connector blocks 52R or 52L to have a stop 64.

The design of the connector blocks also permits the lower connector blocks 54R/54L to shift an additional lateral amount 72R to the right, as shown in FIG. 5D, but this requires some explanation. When moved in this direction, there are no first stops 64 to prevent movement of the lower connector blocks 54R/54L; thus lateral gap 68 will increase to a width greater than W3. However, excessive movement of the patient's mandible to the right will be impeded by virtue of the manner in which the mandible moves. In reality, the mandible does not move in a purely lateral direction (19) with respect to the maxilla, but rather moves in an arcuate direction. This is represented in FIGS. 5C and 5D as arcuate arrows 72L and 72R in the top down views. When a patient moves his mandible to the right as shown in FIG. 5D, the linear design and tight fit between the linear protrusions 58 and 62 will eventually bind under the stress of this arcuate movement, effectively limiting right lateral movement. In other words, when a patient wears MAD 50, he will be able to move his mandible to the right, but not so far as to cause the connector block pairs 52R/54R and 52L/54L to disconnect.

While first stops 64 are preferred to prevent excessive lateral movement of the lower splint 14 with respect to the upper splint 12 in one direction, first stops 64 are not necessarily required in all designs. Just as the linear design of the linear protrusions 58 and 62 in light of slight mandibular arcuate movement will impede excessive lateral movement and disconnection of the splints 12 and 14 in one direction (72R, FIG. 5D), the same is true of movement in the other direction (72L, FIG. 5C). Thus, the connectors blocks may simply have linear protrusions 58 and 62, which may be along the entire lengths W of the blocks or shorter, and no first stops 64.

The combined left 72L and right 72R lateral (but slightly arcuate) movements enabled by the design of MAD 50 are shown in FIG. 5E. Enablement of such lateral movement is beneficial in MAD 50′s design, as this naturally accommodates the motion of the mandible when a patient grinds his teeth. This aids patient comfort, and provides a more natural feel to MAD 50 compared to prior art designs having mechanisms that rigidly connect the upper and lower splints. As a further benefit, MAD 50 prohibits contact and wearing of the teeth should grinding occur.

Another benefit of the design of MAD 50 is its ability to allow a patient (rather than a dentist or orthodontist) to easily adjust the amount of protrusive mandibular advancement that the MAD 50 provides, and this is shown with respects to FIGS. 6A-6F. FIG. 6A is similar to FIG. 5B, and shows the connector block pairs 52R/54R and 52L/54L interlocked with lower blocks 54R/54L completely underlying blocks 52R/52L. This positioning of the lower splint 14 to the upper splint 12 can be said to be at a zero protrusive increment, which is explained further with reference to FIG. 6F. FIG. 6A further shows a cross section of the interlocked connector block pairs taken parallel to their lengths L. Particular linear protrusions 58′ of the upper connector blocks 52L/R are labeled, as are particular linear protrusions 62′ of the lower connector blocks 52L/R. Labeling of these protrusion 58′ and 62′ is helpful in understanding protrusive advancement, as will be shown in subsequent figures. Notice that linear protrusions 62′ are directly behind linear protrusions 58′ at this stage.

Before discussing protrusive advancement, it is also noteworthy that MAD 50 can affect and set the spacing in vertical direction 16 between the upper and lower teeth (and the maxilla and mandible). As shown in the cross section, the amount of vertical spacing (Ttot) is set by the thicknesses T1 of the substrates 56 and 60 used respectively in the upper connector blocks 52R/52L and the lower connector blocks 54R/54L (which can be different); the thicknesses T2 of the linear protrusions 58 and 62 (which can be different); the thickness of the adhesive 66 used to affix the connector blocks to the splints (which can be different); and the thickness of the splint material on the bottom of the upper splint 12 and the top of the lower splint 14 (which can be different). Therefore, the thickness of any of these materials can be changed to achieve a desired vertical spacing.

In FIG. 6B, it is assumed that the patient wishing to increase protrusive mandibular advancement has removed the MAD 50 from his mouth, and has used his hands to move the lower splint 14 to the right (90) of the lower splint 12. This allows the linear protrusions 62 of the lower connector blocks 54R/54L to slide away from the linear protrusions 58 of the upper connector blocks 52R/52L, which results in disconnecting the lower splint 12 from the upper splint 12.

In FIG. 6C, the patient has now advanced (92) the lower splint 14 protrusively forward relative to the upper splint 12 in horizontal direction 18. In FIG. 6D, the patient has then laterally moved the lower splint 14 to the left (94), thus again sliding the linear protrusions 62 in the spaces between the linear protrusions 58, and thus again interlocking the connector block pairs 52R/54R and 52L/54L. As a result, linear protrusions 62′ of the lower connector blocks 54R/54L are now directly in front of the linear protrusions 58′ of the upper connector blocks 52R/52L. This positioning of the lower splint 14 to the upper splint 12 can be said to be at a +1 protrusive increment, which is explained further with reference to FIG. 6F. Note that the distance of protrusive advancement will be determined by the distance between the linear protrusions (L1, FIG. 3), and so a +1 protrusive increment may comprise an advancement of 3 mm according to the example dimensions (L1) provided earlier. Of course, this dimension can be changed to change the amount of incremental advancement.

In FIG. 6E, the steps of FIGS. 6B (disconnection), 6C (advancements), and 6D (re-connection) are repeated. As a result, linear protrusions 62′ of the lower connector blocks 54R/54L are now one space further in front of the linear protrusions 58′ of the upper connector blocks 52R/52L. This positioning of the lower splint 14 to the upper splint 12 can be said to be at a +2 protrusive increment (e.g., 6 mm), which is explained further with reference to FIG. 6F.

It should be noted starting at FIG. 6A that the lower splint 14 can similarly be moved backwards with respect to the upper splint 16 in protrusive increments of −1, −2, etc.

FIG. 6F illustrates the effect in MAD 50 of protrusive advancement of the lower splint 14 (and mandible) relative to the upper splint 12 (and maxilla). The top left of FIG. 6F shows the relative protrusive positioning of a patient's mandible with respect to his maxilla when the patient is not wearing his MAD 50. In this circumstance, the patient's mandible is advanced 0% with respect to a maximum advancement that the mandible could safely be advanced relative to the maxilla. Such a maximum advancement (100%) can be defined for example as an advancement that would put under stress on a patient's ligaments or jaw muscles.

In the top right, the protrusive positioning is shown with the patient wearing MAD 50, and with the lower splint 14 locked to the upper splint 12 as a zero protrusive increment setting. As noted earlier, this setting may be defined as when the upper 52R/52L and lower 54R/54L connector blocks are locked and perfectly overlying each other, as shown in FIG. 6A. Notice at this setting that some baseline amount of therapeutic protrusive advancement is provided by the MAD 50. For example, at this zero protrusive increment setting, the patient's mandible may be advanced at 60% of its potential maximum advancement, which may be sufficient to treat the patient's snoring/OSA symptoms.

In the bottom right, the protrusive positioning of the MAD 50 is shown at a +1 protrusive increment setting. As noted earlier, this setting may be defined when the lower connector blocks 54R/54L are advanced forward one notch from the upper connector blocks 52R/52L, as shown in FIG. 6D. This increases the protrusive advancement from its baseline amount. For example, at this +1 protrusive increment setting, the patient's mandible may be advanced at 75% of its potential maximum advancement, which may be sufficient to treat the patient's snoring/OSA symptoms. If problematic symptoms are still present, a +2 protrusive increment setting may be tried (e.g., 90% of potential maximum advancement), as shown in FIG. 6E, but not shown in FIG. 6F.

The design of MAD 50 is beneficial because the upper and lower splints 12 and 14 can be easily taken apart and put back together again, allowing the patient to experiment with different protrusive increments setting and different degree of protrusive mandibular advancement. However, once a patient has determined a particular protrusive increment/advancement that works for them, they may wish to keep the upper and lower splints 12 and 14 locked into that position so that they cannot easily be taken apart and so that the protrusive increment/advancement is always set in the MAD 50. This may be more convenient because the patient would not have to interlock the upper and lower splints 12 and 14 at the desired protrusive increment/advancement every night when they wish to use the MAD 50. However, for the reasons explained above, it is still desired to fix the upper and lower splints 14 in a manner that allows for at least some degree of lateral movement (19) between the two. Therefore, one may not wish to, for example, rigidly affix the connectors block pairs 52R/54R and 52L/54L at a particular protrusive increment/advancement, using glue for example (although one could do this if desired), because such rigid affixing would not allow for lateral movement.

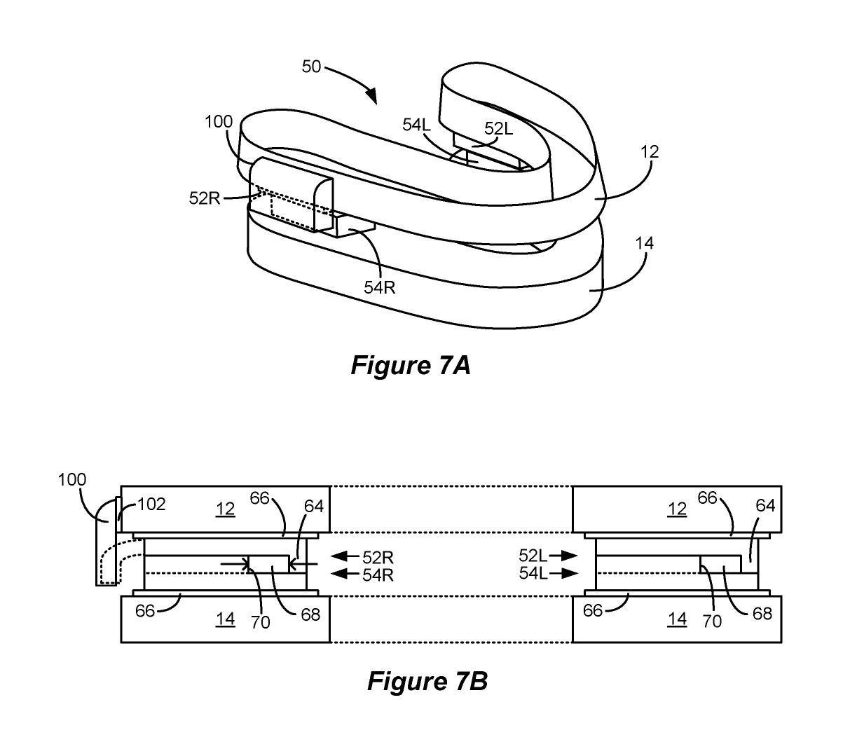

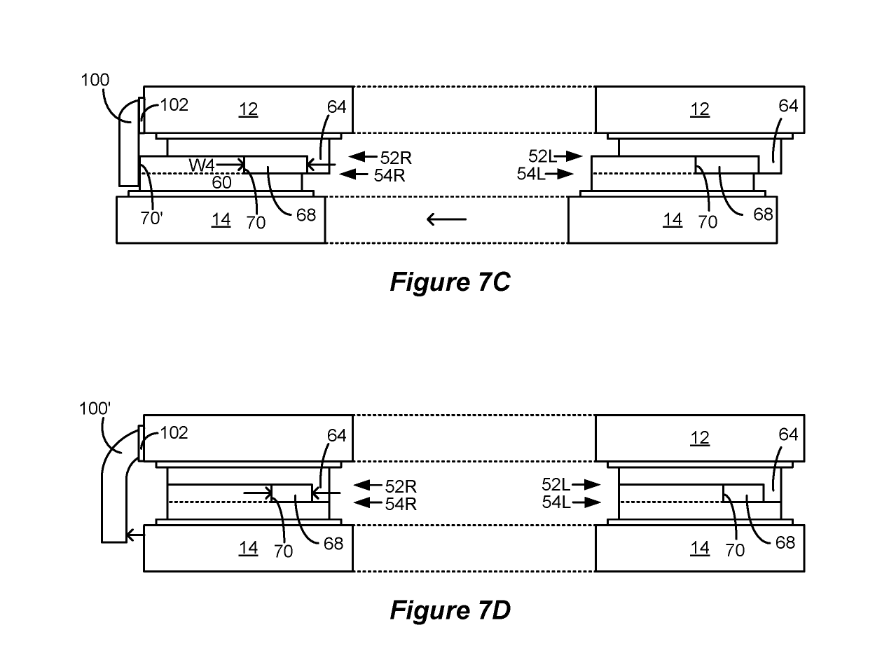

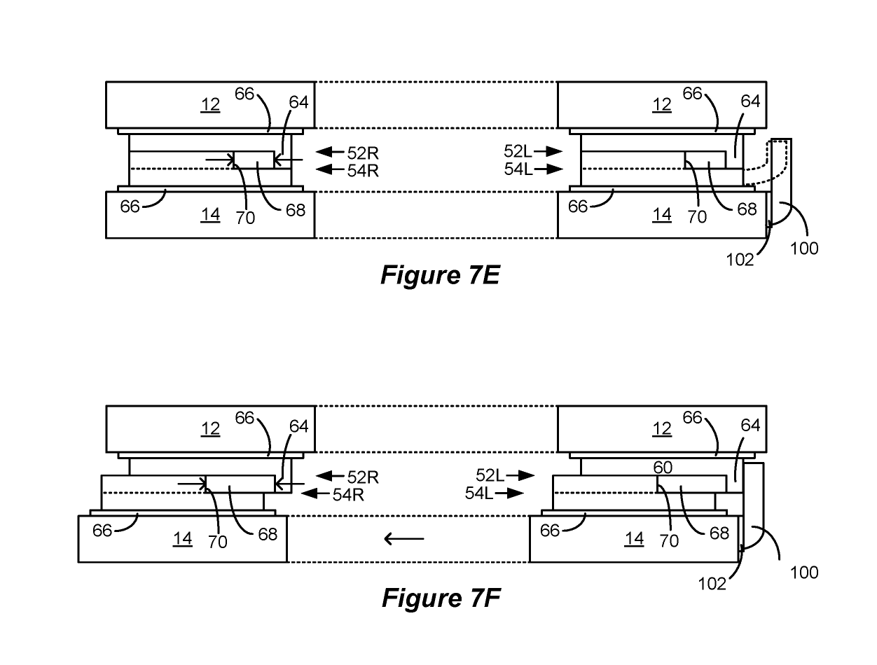

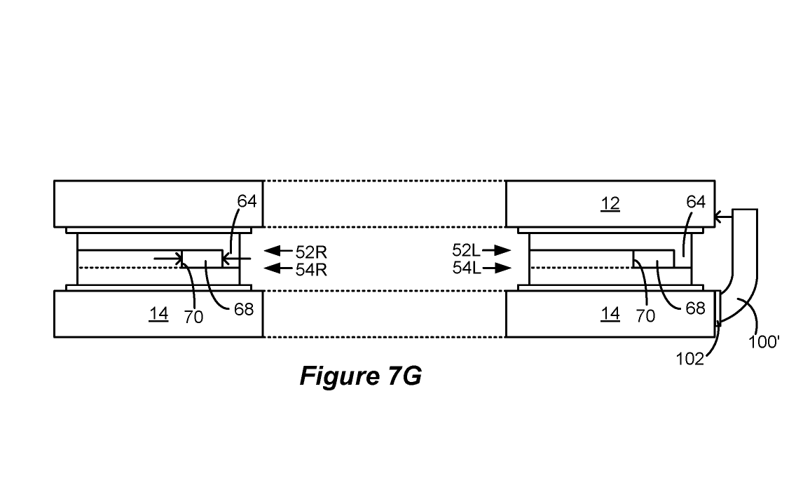

Instead, to affix the splints 12 and 14 together at a particular protrusive increment/advancement in MAD 50 while still permitting later movement between the two, one can instead optionally affix a second stop 100 to one of the lower or upper splints 12 and 14, as shown in FIGS. 7A-7D. As shown in FIG. 7A, the second stop 100 is affixed to the upper splint 12 on the patient's right side, but it may also be affixed to the lower splint 14 (see FIGS. 7E-7G); which splint the second stop 100 is connected to, and on which side it should be connected, will depend on the orientation and position of the connector blocks 52R/52L having the first stops 64—for example, whether first stops 64 are on the right or left edges of the blocks 52R/52L, and whether blocks 52R/52L are connected to the upper or lower splint 12 or 14. It is preferred that the second stop 100 be affixed to the upper or lower splint 12 or 14 on the outside of the splint proximate to a patient's cheek, and that it be made smooth so as not to aggravate the patient's cheek. However, second stop 100 could also be placed on the inside of the splint proximate to the patient's tongue, although this isn't shown. Alternatively, two second stops 100 could be used in both outside and inside locations, for example, outside of right upper connector block 52R, and inside of left upper connector block 52L, although this isn't shown or discussed for simplicity. Two second stops 100 could also be used at left and right outside locations (by combining FIGS. 7B and 7E for example), but with each connected to different ones of the splints 12 and 14, although this also isn't shown.

Second stop 100 may be formed of the same material as the splints or connector blocks (e.g., acrylic) and may comprise a block or mass that is affixed to the relevant splint (e.g., 12) using an adhesive 102, as shown in the cross sections of FIGS. 7B-7D. Adhesive 102 may be similar to the adhesive 66 used to affix the connector blocks 52R/54R and 52L/54L to the splints 12 or 14, which adhesive 102 may again comprise a hard-setting cold-cure acrylic. Alternatively, second stop 100 may be formed entirely of the adhesive 102, which is shaped and cured solid to form the second stop. If a patient decides to have upper and lower splints 12 and 14 affixed together using second stop 100, it would be expected (but not strictly necessary), that affixing of the second stop 100 to the relevant splint would be performed by the dentist or orthodontist, or an associated lab. Preferably, the second stop 100 is affixed to the side of the relevant splint (the generally vertical edge between channel 12C or 14C and flat surface 12F or 14F). However, second stop 100 could also be applied to the side of the connector block (e.g., 52R), as shown in dotted lines in FIG. 7B.

FIG. 7B shows a cross section of the splints and connector blocks after they have been interlocked at a desired protrusive increment/advancement (e.g., FIGS. 6D, 6E), and after the second stop 100 has been applied to lock the splints 12 and 14 together at that position. At this point, the lower splint 14 can only move a lateral amount 72L to the left, as described earlier with respect to FIG. 5C, before the faces 70 of the linear protrusions 62 of the lower connector blocks 54R/54L come into contact with the first stops 64 of the upper connector blocks 52R/52L. In other words, the connector blocks pairs 52R/54R and 52L/54L cannot become disconnected by such left lateral movement, and hence the lower splint 14 will stay interlocked with the upper splint 12 at the prescribed protrusive position.

FIG. 7C shows the result of lateral movement of the lower splint 14 to the right of the upper splint 12 after the second stop 100 is connected. Without such a second stop 100, such right lateral movement would cause the lower splint 14 to slide away from and disconnect from the upper splint 12 (see, e.g., FIG. 6B). But when second stop 100 has been affixed, opposing faces 70′ of the linear protrusions 62 of (in this example) lower connector block 54R, and/or its substrate 60, come into contact with the second stop 100. At this stopped position, the lateral gap 68 may have a longer width (W4) than the patient can otherwise effect by rightward movement of his mandible (see FIG. 5D; 72R) when the MAD 50 in the patient's mouth. Nonetheless, at the stopped position in FIG. 7C, the linear protrusions 58 and 62 of the blocks are still interlocked, and therefore the lower splint 14 cannot move in a horizontal direction 18 or in a vertical direction 16 with respect to the upper splint 12. In other words, the connector block pairs, and the upper and lower splints 12 and 14, are effectively locked together by the second stop 100, while still allowing some degree of left and right lateral movement. This allows the patient to insert and removed the MAD 50 into his mouth as one connected piece, and with the desired protrusive increment/advancement, without having to slide the upper and lower splits 12 and 14 together and apart each time the MAD 50 is used.

FIG. 7D shows an alternative of the second stop 100′ that can be affixed to the relevant splint (e.g., 12) once a desired protrusive increment/advancement has been set. In this example, the second stop 100′ is designed to prevent excessive right lateral movement of the lower splint 14 by causing the lower splint 14 itself to contact the second stop 100′ (see arrow), rather than by causing the lower connector block 54R contacting the second stop 100′. The effect however is the same as in FIG. 7C, as the stopped position of FIG. 7D will also prevent the upper and lower splints 12 and 14 from disconnecting.

As noted earlier, where it is logical to connect the second stops 100 or 100′ depends on the orientation and position of the connector blocks 52R/52L having the first stops 64, which can vary as described earlier. However, even if the orientation and position of the blocks are not altered, the second stop 100 or 100′ can still be positioned at different locations. As shown in FIGS. 7E-7G, the second stop 100 or 100′ is connected to outer edge of the lower splint 14 on the patient's left side. Such positioning of the second stop 100 or 100′ in FIGS. 7E-7G also prevents excessive right lateral movement of the lower splint 14 with respect to the upper splint 12, and so keep the two splints interlocked at the desired protrusive increment/advancement. (Splints 12 and 14 cannot disconnect by virtue of left lateral movement of the lower splint 14 for the reasons explained earlier). FIG. 7F shows operation of second stop 100. When the lower splint 14 is moved laterally to the right, the second stop 100 contacts upper left connector block 52L, and more particularly eventually hits the outward facing edge of the first stop 64 and/or its substrate 60. FIG. 7G shows operation of second stop 100′ which stops right lateral movement because the second stop 100′ will eventually hit the upper splint 12 (rather than connector block 52L). In any event, and similarly to FIGS. 7A-7D, the MAD 50 design of FIGS. 7E-7G permit some degree of left and right lateral movement of the lower splint 14 relative to the upper splint 12 without causing the upper and lower splints 12 and 14 to disconnect, even when taken out of the patient's mouth.

Application of second stops 100 or 100′ provide an essentially permanent affixation of the upper and lower splints 12 and 14 at a particular protrusive increment/advancement in MAD 50. However, should a different protrusive position be desired later, the second stops 100 or 100′ can easily be cut away and filed down if necessary by the dentist, orthodontist, or lab, thus allowing the now disconnected upper and lower splints 12 and 14 to be readily connected and disconnected as described earlier. If a new protrusive increment/advancement setting is determined, the upper and lower splints 12 and 14 may be connected at that position, and one or more new second stops 100 or 100′ applied if desired.

Although particular embodiments of the present invention have been shown and described, it should be understood that the above discussion is not intended to limit the present invention to these embodiments. It will be obvious to those skilled in the art that various changes and modifications may be made without departing from the spirit and scope of the present invention. Thus, the present invention is intended to cover alternatives, modifications, and equivalents that may fall within the spirit and scope of the present invention as defined by the claims.

What is claimed is:

1. A Mandibular Advancement Device (MAD), comprising:
an upper splint configured to receive at least some of a patient's upper teeth;
a lower splint configured to receive at least some of the patient's lower teeth;
right and left upper connector blocks connected to the upper splint; and
right and left lower connector blocks connected to the lower splint,
wherein the right upper and lower connector blocks are configured to interlock as a right pair, and the left upper and lower connector blocks are configured to interlock as a left pair, by sliding the lower splint in a lateral direction with respect to the upper splint.

2. The MAD of claim 1, wherein the right and left upper connector blocks are connected to the upper splint, and the right and left lower connector blocks are connected to the lower splint, using an adhesive.

3. The MAD of claim 1, wherein the interlocked right and left pairs permit the lower splint to move in the lateral direction relative to the upper splint, but prohibit the lower splint from moving in a horizontal direction relative to the upper splint.

4. The MAD of claim 3, wherein the interlocked right and left pairs prohibit the lower splint from moving in a vertical direction relative to the upper splint.

5. The MAD of claim 1, wherein the right and left pairs are configured to interlock at a plurality of different protrusive increments, thus setting different protrusive advancements of the lower splint with respect to the upper splint.

6. The MAD of claim 1, wherein the right upper and lower connector blocks and the left upper and lower connector blocks are planar, wherein the right and left upper connector blocks are located in a first plane, wherein the right and left lower connector blocks are located in a second plane, and wherein the first and second planes are parallel when the right and left pairs are interlocked.

7. The MAD of claim 1, wherein the right and left upper connector blocks are connected to the upper splint under where a patient's right and left molar and/or pre-molar upper teeth would be received within the upper splint, and wherein the right and left lower connector blocks are connected to the lower splint above where a patient's right and left molar and/or pre-molar lower teeth would be received within the lower splint.

8. The MAD of claim 7, wherein the right and left upper connector blocks are connected to a flat surface of the upper splint, wherein the right and left lower connector blocks are connected to a flat surface of the lower splint, wherein the flat surfaces of the upper and lower splints face each other when the right and left pairs are interlocked.

9. The MAD of claim 1, wherein the right and left upper connector blocks, and the right and left lower connector blocks, each comprise a plurality of linear protrusions parallel to the lateral direction and spaced in a horizontal direction.

10. The MAD of claim 9, wherein the plurality of linear protrusions have a trapezoidal cross section in the horizontal direction.

11. The MAD of claim 9, wherein the right pair, and the left pair, are configured to interlock by sliding the lower splint in the lateral direction with respect to the upper splint such that the plurality of linear protrusions of the right pair, and the plurality of linear protrusions of the left pair, interlock.

12. The MAD of claim 11, wherein the interlocking linear protrusions of the right pair, and the interlocking linear protrusions of the left pair, prohibit the lower splint from moving in the horizontal direction and a vertical direction relative to the upper splint.

13. The MAD of claim 11, wherein the interlocking linear protrusions of the right pair, and the interlocking linear protrusions of the left pair, permit the lower splint to move in the lateral direction relative to the upper splint.

14. The MAD of claim 13, wherein the upper right and left connector blocks, or the lower right and left connector blocks, comprise first stops to prohibit excessive movement of the lower splint relative to the upper splint in the lateral direction.

15. The MAD of claim 14, wherein the first stops are perpendicular to the plurality of linear protrusions.

16. The MAD of claim 11, wherein the plurality of linear protrusions of the right pair, and the plurality of linear protrusions of the left pair, are configured to interlock at a plurality of different protrusive increments, thus setting different protrusive advancements of the lower splint with respect to the upper splint.

17. The MAD of claim 1, further comprising at least one second stop affixed to the upper or lower splint, wherein the at least one second stop prohibits the right and left pairs from becoming disconnected once interlocked.

18. A Mandibular Advancement Device (MAD), comprising:
an upper splint configured to receive at least some of a patient's upper teeth;
a lower splint configured to receive at least some of a patient's lower teeth;
right and left upper connector blocks connected to the upper splint; and
right and left lower connector blocks connected to the lower splint,
wherein the right and left upper connector blocks, and the right and left lower connector blocks, each comprise a plurality of linear protrusions parallel to a lateral direction and spaced in a horizontal direction,
wherein the plurality of linear protrusions of the right upper and lower connector blocks, and the plurality of linear protrusions of the left upper and lower connector blocks, are configured to interlock as right and left pairs respectively.

19. The MAD of claim 18, wherein the right and left upper connector blocks are connected to the upper splint, and the right and left lower connector blocks are connected to the lower splint, using an adhesive.

20. The MAD of claim 18, wherein the interlocked right and left pairs permit the lower splint to move in the lateral direction relative to the upper splint, but prohibit the lower splint from moving in the horizontal direction relative to the upper splint.

21. The MAD of claim 20, wherein the interlocked right and left pairs prohibit the lower splint from moving in a vertical direction relative to the upper splint.

22. The MAD of claim 18, wherein the right and left pairs are configured to interlock at a plurality of different protrusive increments, thus setting different protrusive advancements of the lower splint with respect to the upper splint.
